(19) United States
(12) Patent Application Publication
(10) Pub. No.: US 2017/0157369 A1
(43) Pub. Date: Jun. 8, 2017

(54) MEDICAL APPARATUSES AND RELATED METHODS FOR INTRANASAL FLUID DELIVERY

(71) Applicant: Daniel Scott Faith, Fayetteville, GA (US)

(72) Inventor: Daniel Scott Faith, Fayetteville, GA (US)

(21) Appl. No.: 14/957,509

(22) Filed: Dec. 2, 2015

(51) Int. Cl. A61M 25/10; A61M 19/00; A61M 11/00

(52) U.S. Cl. CPC A61M 25/1009; A61M 11/00; A61M 19/00; A61M 25/10181; A61M 25/10185; A61M 2025/1052; A61M 2025/1079; A61M 2210/0618; A61M 2210/0681; A61M 2210/1046

(57) ABSTRACT

A method includes blocking a posterior opening of a nasal cavity of a patient and delivering a medical fluid through a catheter to a sphenopalatine ganglion of the patient after blocking the posterior opening of the nasal cavity.

### TECHNICAL FIELD

This disclosure relates to apparatus and methods for intranasal fluid delivery, in particular, for intranasal medical fluid delivery for treatment of neurological diseases.

### BACKGROUND

In the human anatomy, the sphenopalatine ganglion is a group of nerves located in the superior meatus, which is a nasal passage positioned within the nasal cavity superior to the middle turbinate. The sphenopalatine ganglion includes nerves for the sympathetic, parasympathetic, and sensory nervous systems. The sphenopalatine ganglion is covered by the nasal mucosa.

Abnormalities of the sphenopalatine ganglion can cause increased sympathetic activity, leading to severe pain. To manage the pain, a physician can perform a nerve block procedure on the sphenopalatine ganglion. This procedure can include delivering an anesthetizing agent to the sphenopalatine ganglion to inhibit the ability of the sphenopalatine ganglion from transmitting pain signals.

### SUMMARY

In one aspect of this disclosure, a medical apparatus includes a catheter body and an expandable member. The catheter body has a medical fluid lumen. The catheter body includes an insertion end region configured to be inserted into a nasal cavity of a patient. A lateral portion of the insertion end region of the catheter body defines a side opening in fluid communication with the medical fluid lumen of the catheter body. The expandable member is secured to a portion of the catheter body distal to the side opening. The expandable member is configured to block a posterior opening of the nasal cavity of the patient when the insertion end region of the catheter body is positioned within the nasal cavity of the patient and the expandable member is expanded. The medical fluid lumen is configured to be placed in fluid communication with a source of the medical fluid such that the medical fluid can be passed through the medical fluid lumen and exit the side opening to contact a sphenopalatine ganglion of the patient when the side opening is positioned within the nasal cavity of the patient.

In some examples, the medical apparatus can include a stabilizing member securable to the catheter body and sized and dimensioned to inhibit the stabilizing member from entering the nasal cavity of the patient through an anterior naris of the nasal cavity of the patient. The stabilizing member can be positionable against the nose of the patient. The stabilizing member can be slidable along the catheter body. The stabilizing member can be lockable in a position along the catheter body such that, when the expandable member blocks the posterior opening and the stabilizing member is positioned against the nose of the patient and locked, the stabilizing member inhibits distal movement of the expandable member from the posterior opening. The stabilizing member can include a disc formed of a soft material, the disc having a diameter greater than a diameter of the anterior naris of the nasal cavity.

In some examples, the medical apparatus can include an orientation indicator disposed near a manipulation end of the catheter body. The orientation indicator can be indicative of an angular position of the side opening relative to a longitudinal axis of the catheter body. The medical can apparatus can include a hub positioned at the manipulation end of the catheter body and through which the medical fluid lumen extends. The hub can include the orientation indicator.

In some examples, the medical apparatus can include a radiopaque marker disposed along the catheter body distal to the expandable member. The medical apparatus can include a radiopaque marker disposed along the catheter body proximal to the expandable member.

In some examples, the expandable member can include an inflatable balloon configured to be placed in fluid communication with a source of gas such that the gas can be passed through the catheter body to inflate the inflatable balloon. The insertion end region of the catheter body can include a distal wall. The distal wall can prevent the gas from exiting the catheter body at a position distal to the inflatable balloon and the medical fluid from exiting the medical fluid lumen at a position distal to the side opening. The inflatable balloon can be inflatable to a volume of at least 1.75 cubic centimeters such that the inflatable balloon, when inflated, is configured to form a substantially fluid-tight seal between the nasal cavity and a nasopharynx of the patient. The medical apparatus can include a gate valve connected to a proximal end of the catheter body. The gate valve can be closeable to prevent the gas within the inflatable balloon from escaping the inflatable balloon.

In some examples, the medical apparatus can include a distance marker disposed on the catheter body at a distance from a distal end of the catheter body approximately equal to a distance between an anterior naris of the patient and a nasopharynx of the patient. The distance marker can be approximately eight to twelve centimeters proximal from a distal end of the catheter body.

In some examples, the side opening includes a slit sized and dimensioned to cause the medical fluid ejected from the slit to foam.

In a further aspect, a method includes blocking a posterior opening of a nasal cavity of a patient and delivering a medical fluid through a catheter to a sphenopalatine ganglion of the patient after blocking the posterior opening of the nasal cavity.

In some examples, blocking the posterior opening of the nasal cavity can include expanding an expandable member to block the posterior opening of the nasal cavity. The method can further include stabilizing the expandable member within the posterior opening of the nasal cavity. Stabilizing the expandable member within the posterior opening of the nasal cavity can include stabilizing the catheter against a nostril of the patient.

In some examples, blocking the posterior opening of the nasal cavity can include advancing an expandable member into the posterior opening of the nasal cavity before expanding the expandable member to block the posterior opening of the nasal cavity. Advancing the expandable member into the posterior opening of the nasal cavity can include advancing the catheter into the posterior opening of the nasal cavity.

In some examples, blocking the posterior opening of the nasal cavity can include expanding an expandable member to block the posterior opening of the nasal cavity, and expanding the expandable member to block the posterior opening of the nasal cavity can include expanding the expandable member in a nasopharynx of the patient. The method can include, after expanding the expandable member, retracting the expandable member away from the nasopharynx toward the posterior opening to apply a force to tissue defining the posterior opening. Retracting the expandable member away from the nasopharynx toward the posterior opening can cause the expandable member to reduce flow of fluid from the nasal cavity into the nasopharynx. Retracting the expandable member away from the nasopharynx toward the posterior opening can cause the expandable member to form a substantially fluid-tight seal around the posterior opening. The fluid-tight seal can separate the nasal cavity and the nasopharynx of the patient.

In some examples, blocking the posterior opening of the nasal cavity can include expanding an expandable member to block the posterior opening of the nasal cavity, and the method can further include, after delivering the medical fluid, contracting the expandable member and removing the expandable member from the patient. The method can further include expanding the expandable member includes injecting at least 1.75 cubic centimeters of air into an inflatable balloon, and contracting the expandable member includes deflating the inflatable balloon. Inflating the inflatable balloon can further include locking a valve connected to the inflatable balloon. Deflating the inflatable balloon can include unlocking the valve connected to the inflatable balloon and releasing the at least 1.75 cubic centimeters of air. The method can include, before contracting the expandable member and removing the expandable member from the patient and after delivering the medical fluid, waiting at least five minutes for trans-mucosal absorption of the medical fluid.

In some examples, blocking the posterior opening of the nasal cavity can include expanding an expandable member to block the posterior opening of the nasal cavity, and inserting the catheter into the nasal cavity can include advancing the expandable past an anterior opening of the nasal cavity by at least ten centimeters such that the expandable member advances into a nasopharynx of the patient. Expanding the expandable member of the catheter to block the posterior opening of the nasal cavity can include expanding the expandable member within the nasopharynx of the patient.

In some examples, delivering the medical fluid through the catheter to the sphenopalatine ganglion of the patient can include dispensing the medical fluid in a lateral direction from the catheter. The method can further include, before dispensing the medical fluid in the lateral direction from the catheter, rotating the catheter such that the lateral direction is directed toward the sphenopalatine ganglion.

In some examples, the medical fluid can include an anesthetic. Delivering the medical fluid to the sphenopalatine ganglion of the patient can include delivering the anesthetic to the sphenopalatine ganglion to perform a nerve block of the sphenopalatine ganglion. The method can further include diagnosing the patient as having a neurological disease affecting the sphenopalatine ganglion. The neurological disease can include chronic migraines.

In some examples, delivering the medical fluid can include delivering a spray of the medical fluid. Delivering the spray of the medical fluid can include covering nasal mucosa covering the sphenopalatine ganglion with the medical fluid.

In some examples, the medical fluid can include a foam. The medical fluid can include a viscous foam. Delivering the medical fluid to the sphenopalatine ganglion can include filling the nasal cavity with the foam.

In some examples, the medical fluid can include a gel.

The medical apparatuses, devices, systems, and methods of this disclosure provide the following advantages. When using an example of the medical apparatus described herein, an operator can—instead of, for example, moving and directing the insertion end of the catheter body of the medical apparatus into the superior meatus of a patient to reach the sphenopalatine ganglion—simply move the insertion end of the catheter body past the superior meatus and generally direct the side opening of the catheter body toward the sphenopalatine ganglion. In some cases, the physiology of the patient can include structures within the nasal cavity or abnormalities affecting the nasal cavity that can increase tortuosity of a path from the anterior opening of the nasal cavity to the sphenopalatine ganglion. The operator using the medical apparatus can avoid the complex path formed by these abnormalities, which can include nasal septal deviation, concha bullosa, nasal congestion, and polyps. The operator can perform the nerve block of the sphenopalatine ganglion without having to precisely guide the insertion end into the superior meatus. The side opening can remain in a portion of the nasal cavity outside of the bony structures defining the superior meatus and surrounding the sphenopalatine ganglion so the operator can avoid complex maneuvers of the medical apparatus within the nasal cavity.

The medical apparatus can enable localization of the medical fluid within a target area within the nasal cavity of the patient. In addition to including the side opening for the targeted application of the medical fluid toward the sphenopalatine ganglion, the medical apparatus can reduce leakage of the medical fluid into portions of the anatomy of the patient outside of the nasal cavity, such as the nasopharynx. In particular, the operator can block openings in the nasal cavity, such as the posterior naris of the nasal cavity, to impede the flow of the medical fluid toward regions outside of the nasal cavity. The operator can use the expandable member to form the seal along the openings of the nasal cavity and thereby maintain the medical fluid within the nasal cavity.

Because the medical fluid can be retained within the nasal cavity, the operator can also dispense the medical fluid to cover a larger portion of the nasal cavity. The risk for the medical fluid, which can contain the numbing agent or anesthetic, to anesthetize collateral regions of the anatomy, can be reduced even when the medical fluid is dispensed to cover the larger portion of the nasal cavity.

In addition, retention of the medical fluid within the nasal cavity can enable the medical fluid to remain within the nasal cavity for a greater period of time. While performing the nerve block of the sphenopalatine ganglion, the operator can provide a greater waiting period following the delivery of the medical fluid because the medical fluid does not migrate out of the nasal cavity. As a result, the operator can provide more time for the active compound in the medical fluid to be absorbed through the nasal mucosa toward the sphenopalatine ganglion.

The details of one or more implementations are set forth in the accompanying drawings and the description below. Other features and advantages will be apparent from the description and drawings, and from the claims.

### DESCRIPTION OF DRAWINGS

FIG. 1 illustrates a catheter assembly inserted into a nasal cavity of a patient.

FIG. 2A illustrates the catheter assembly that is shown disposed within the patient's nasal cavity in FIG. 1.

FIG. 2B is a side view of a distal portion of the catheter assembly of FIG. 2A.

FIG. 2C is a side view of a portion of the catheter assembly of FIG. 2A including a stabilizing member.

FIG. 2D is a cross-sectional view of the catheter assembly taken along the section line 2D-2D shown in FIG. 2C.

FIGS. 3A to 3H depict various operations using a catheter assembly.

FIG. 4 is a side view of a distal portion of another example of a catheter assembly.

### DETAILED DESCRIPTION

This disclosure relates to a medical apparatus, such as a catheter assembly having a medical fluid lumen, that can administer a medical fluid including an active compound (e.g., a drug, an anesthetic, or other appropriate medicament) to a patient through a nasal cavity of the patient. The catheter assembly can be inserted into the nasal cavity of the patient, and the medical fluid can be delivered through the medical fluid lumen to the nasal cavity. In particular, the medical fluid can be directed toward the nasal mucosa covering the sphenopalatine ganglion of the patient. The active compound can be, for example, an anesthetic or numbing agent such as lidocaine. When the medical fluid including the active compound contacts the sphenopalatine ganglion of the patient, the active compound can cause nervous system signals sent along the sphenopalatine ganglion to be interrupted.

During a sphenopalatine ganglion nerve block procedure, the patient can lie in the supine position while an operator administers the medical fluid containing the anesthetic into the nasal cavity of the patient. During administration of the medical fluid, because of gravity and the geometry of the nasal cavity, the medical fluid can drain toward a posterior region of the nasal cavity. In some cases, the medical fluid can drain toward the nasopharynx immediately posterior to the nasal cavity. Medical apparatuses, catheter assemblies, and methods to use these apparatuses and assemblies described herein can block the posterior opening of the nasal cavity to inhibit leakage of the medical fluid from the nasal cavity into the nasopharynx. These apparatuses, catheter assemblies, and methods can facilitate localization of the medical fluid such that the medical fluid remains in the target area, e.g., the region surrounding the sphenopalatine ganglion within the nasal cavity.

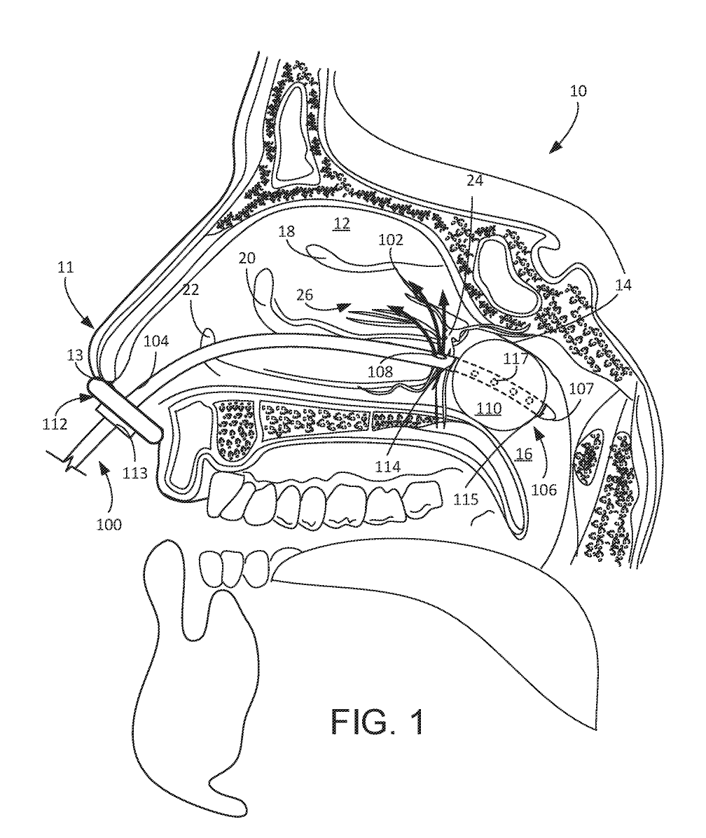

FIG. 1 schematically depicts a sagittal cross-section of the head 10 of a patient. The cross-section of FIG. 1 goes through a plane passing through the nose 11 and one nasal cavity 12 (of the right and left nasal cavities) as it would typically appear in the human anatomy. The two nasal cavities in humans are generally symmetric about the median plane of the human body. The nasal cavity 12 includes anterior and posterior openings. In particular, the anterior naris 13 is the anterior opening to the nasal cavity 12 and is an external opening forming a path from outside of the human body into the nasal cavity 12. The posterior naris 14 is the posterior opening to the nasal cavity 12 and is an interior opening connecting the nasopharynx 16 to the nasal cavity 12.

The nasal cavity 12 is lined with nasal mucosa defining the nasal cavity 12 and covering bony structures supporting the nasal cavity 12. The superior sinus turbinate 18, the middle sinus turbinate 20, and the inferior sinus turbinate 22 are curved bony protrusions located within the nasal cavity 12. The sphenopalatine ganglion 24 is located in the superior meatus 26 defined by the middle sinus turbinate 20 and the superior sinus turbinate 18. Schematically depicted in FIG. 1, the sphenopalatine ganglion 24 is typically covered by the nasal mucosa.

### Catheter Assembly

An example of a medical apparatus to perform a nerve block of the sphenopalatine ganglion 24 is shown in FIG. 1. The medical apparatus is a catheter assembly 100, which is shown dispensing a medical fluid 102 toward the sphenopalatine ganglion 24 to perform the nerve block of the sphenopalatine ganglion 24. The nerve block, also known as a regional nerve blockade, interrupts nervous system signals sent through the sphenopalatine ganglion 24. The interruption of these signals can reduce pain signals associated with the sphenopalatine ganglion 24. These pain signals may be the result of a neurological disease, such as chronic migraines, cluster headache, trigeminal neuralgia, herpes zoster, paroxysmal hemicrania, cancer of the head or neck, facial pain that is atypical, complex regional pain syndrome (CRPS), temporomandibular disorder, nasal contact point, and headache vasomotor rhinitis. The medical apparatus 100 can also treat other neurological diseases or other medical conditions for patients who can benefit from improved wellbeing or comfort through application of medication to the sphenopalatine ganglion. The medical apparatus 100 can treat the neurological disease by reducing these pain signals.

The medical fluid 102 includes a medication or drug, for example, an anesthetic, corticosteroid, or numbing agent that blocks the sphenopalatine ganglion 24. These active compounds in the medical fluid 102, when delivered to the sphenopalatine ganglion 24, can reduce the ability of nerves, such as those of the sphenopalatine ganglion 24, from transmitting signals.

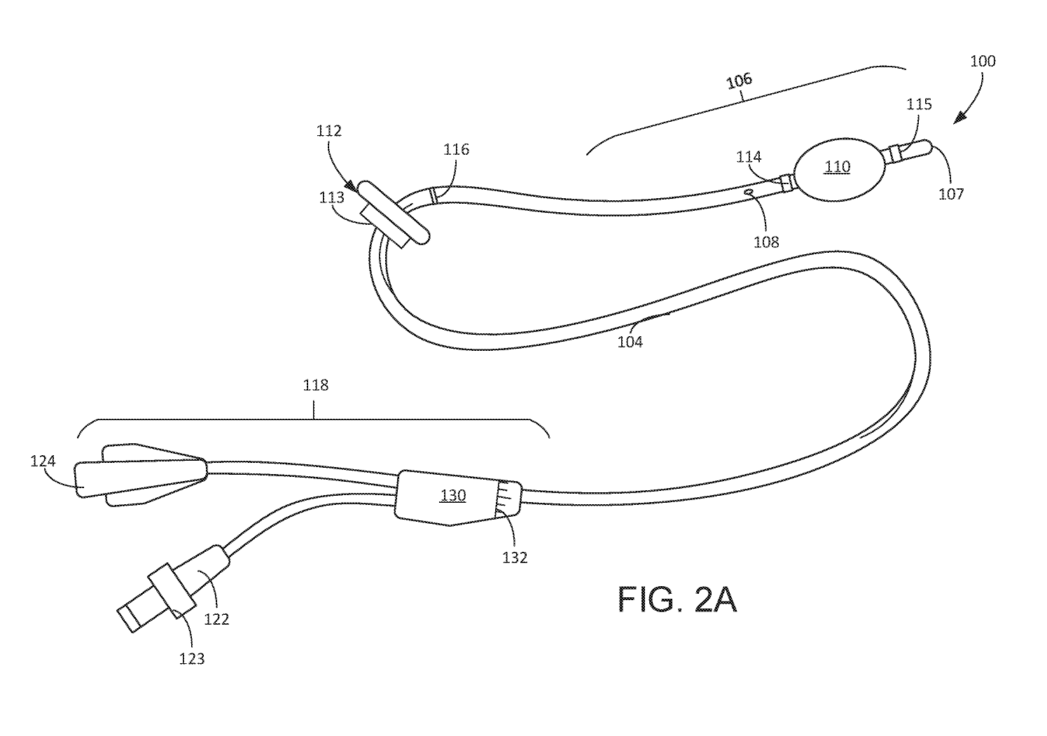

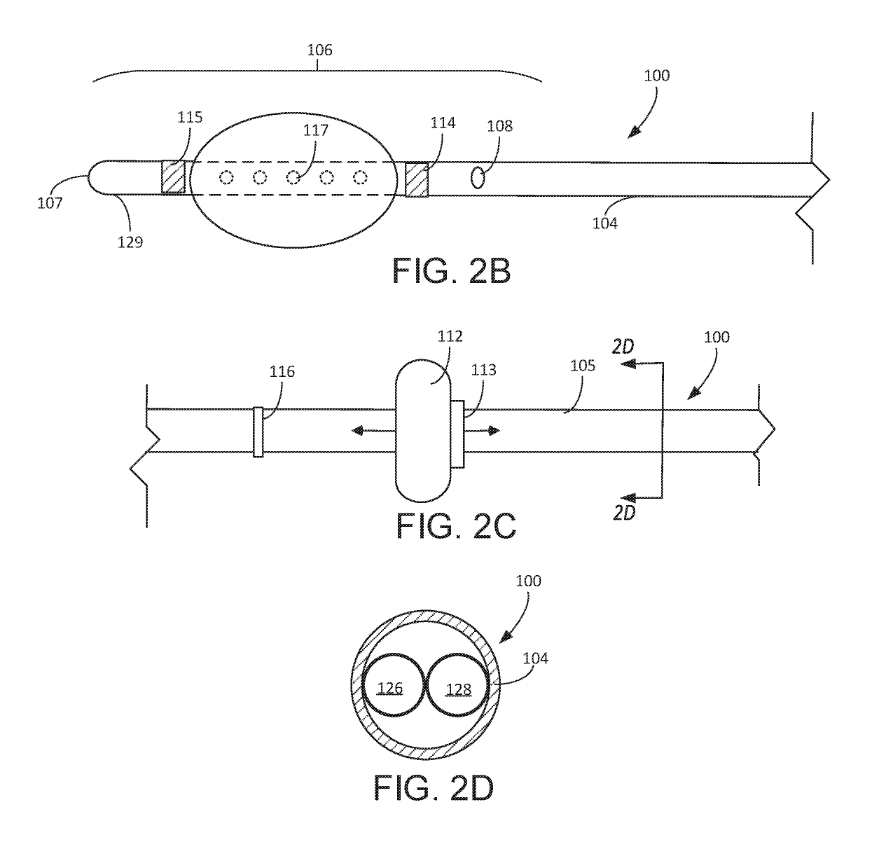

Referring to FIGS. 1 and 2A, the catheter assembly 100 includes a catheter body 104 having an insertion end region 106 positioned toward an insertion end 107. In the context of the description for the example of the catheter assembly 100 and other examples described herein, the distal direction refers to the direction along a length of the catheter assembly 100 pointing away from an operator of the catheter assembly 100 toward a patient during a treatment. The proximal direction refers to the opposite direction along the length of the catheter assembly 100 pointing toward the operator from the patient.

Along the insertion end region 106, the catheter body 104 includes an opening 108 from which the medical fluid 102 is dispensed. The opening 108 can be a side hole of the catheter assembly 100 in which the opening 108 is defined by a lateral portion of the catheter body 104 in the insertion end region 106. By being on the lateral portion of the catheter body 104, the opening 108 can be facing a direction perpendicular to a longitudinal axis of the catheter body 104. The opening 108 of the catheter assembly 100 is proximal to the insertion end 107. The opening 108 can have a diameter between, for example, 1 millimeter and 2 millimeters (e.g., approximately 1.5 millimeters). The catheter body 104 can have a diameter between, for example, 1 millimeter and 3 millimeters (e.g., approximately 1.5 millimeters, or between French size 3 and 6).

During the sphenopalatine ganglion nerve block treatment, the catheter assembly 100 can enable targeted delivery of the medical fluid 102 to the sphenopalatine ganglion 24 within the nasal cavity 12. As described in greater detail herein, the catheter assembly 100 can localize the medical fluid 102 to the nasal cavity 12 of the patient. In particular, the catheter assembly 100 can keep the medical fluid 102 in the vicinity of the targeted sphenopalatine ganglion 24 and reduce the medical fluid 102 that leaves the nasal cavity 12 through the posterior naris 14 into the nasopharynx 16.

During the treatment, an expandable member 110 positioned at the insertion end region 106 of the catheter body 104 can be expanded in the nasopharynx 16 and retracted to block the posterior naris 14. As shown in FIG. 2B, which depicts the insertion end region 106 of the catheter assembly 100, the expandable member 110 is secured to the catheter body 104. The opening 108 can be positioned proximal to the expandable member 110 so that, when the expandable member 110 is positioned in the posterior naris 14, the opening 108 is positioned within the nasal cavity 12. The opening 108 can be positioned, for example, 5 millimeters to 15 millimeters (e.g., approximately 10 millimeters) proximal to a proximal end of the expandable member 110.

The expandable member 110 is compliant so that it can conform to the anatomy of the nasopharynx 16 and the posterior naris 14 and form a seal against the posterior naris 14 of the nasal cavity 12. In the example depicted in FIGS. 2A to 2D, the expandable member 110 of the catheter assembly 100 is an inflatable balloon that expands upon delivery of gas to the expandable member 110. The inflatable balloon can be an elastomeric or rubber material, such as latex, polyurethane, or silicone rubber.

The expandable member 110 can be attached to the catheter body 104 such that a seal is formed between the catheter body 104 and the expandable member 110. For example, the expandable member 110 can be thermally bonded, ultrasonically welded, etc. Gas delivered to the expandable member 110 thus remains within the expandable member 110 and does not escape through the interface between the expandable member 110 and the catheter body 104. The expandable member 110 can be operated to be contracted to an unexpanded state or to be expanded to an expanded state. In the unexpanded state, the expandable member 110 has a size or can be compressed to a size less than the sizes of the anterior naris 13 and the posterior naris 14. For example, if the expandable member 110 is an inflatable balloon, the outer diameter of the inflatable balloon in the unexpanded state is less than the diameters of the anterior naris 13 and the posterior naris 14. The expandable member 110 can thus easily move past the anterior naris 13 and into the posterior naris 14 in the unexpanded state.

In the expanded state, the expandable member 110 is expanded to a size larger than the size of the posterior naris 14. The size of the expandable member 110 in the expanded state can have a size dependent on the size, age, or other relevant characteristics of the patient.

If the expandable member 110 is an inflatable balloon, the inflatable balloon in the expanded state can have a volume between, for example, 2 cubic centimeters and 10 cubic centimeters (e.g., between 2 cubic centimeters and 5 cubic centimeters, 5 cubic centimeters and 10 cubic centimeters, 4.2 cubic centimeters and 8.2 cubic centimeters, 5 cubic centimeters and 7 cubic centimeters, 2.5 cubic centimeters and 3.5 cubic centimeters, 2.75 cubic centimeters and 3.25 cubic centimeters, approximately 4 cubic centimeters, approximately 6 cubic centimeters, or approximately 8 cubic centimeters). In some cases, the expandable member 110 in the expanded state has a volume that is at least 1.75 cubic centimeters. The expandable member 110 in the expanded state can have a size, for example, 105% to 125% the size of the posterior naris 14. In some cases, the expandable member 110 in the expanded state has a width that is at least 1 centimeter. In some examples, the expandable member 110 in the expanded state has a width between 1.5 centimeters and 3 centimeters (e.g., between 2.6 centimeters and 2.9 centimeters or 2.7 centimeters and 2.8 centimeters).

As shown in FIGS. 2A and 2C, a stabilizing member 112, movable along the catheter body 104 and positioned proximal to the expandable member 110, supports the catheter assembly 100 against the anterior naris 13. The stabilizing member 112 is secured or securable to the catheter body 104 such that the stabilizing member 112 can slide along the catheter body 104. In particular, the stabilizing member 112 can be slid so that it is positioned against the nose 11 of the patient.

As depicted in FIG. 2C, which shows a region of the catheter assembly 100 where the stabilizing member 112 is located, the stabilizing member 112 can further include a locking member 113 that locks the stabilizing member 112 in place along the catheter body 104. The locking member 113, in some cases, is a rotatable member that, when rotated, further engages the stabilizing member 112 to decrease an inner diameter of the stabilizing member 112. The locking member 113, for example, can be a nut that compresses threaded arms of the stabilizing member 112 against the catheter body 104 as the locking member 113 is screwed onto the threaded arms. The decreased diameter can increase friction force between the stabilizing member 112 and the catheter body 104 so that that the stabilizing member 112 can maintain its position along the catheter body 104.

The stabilizing member 112 maintains a position of the catheter assembly 100 within the nasal cavity 12 when the expandable member 110 has expanded and is blocking the posterior naris 14. After being expanded and retracted into the posterior naris 14, the expandable member 110 may experience a force that tends to push the expandable member 110 in a distal direction away from the nasal cavity 12. The force can cause movement of, in addition to the expandable member 110, the rest of the catheter assembly 100 relative to the nasal cavity 12. By pushing against the nose 11 and the anterior naris 13, the stabilizing member 112 experiences a proximally directed traction force opposite of the distally directed force on the expandable member 110 from the posterior naris 14. The locking member 113, when locked, enables the stabilizing member 112 to be locked into its position along the catheter body 104. When the expandable member 110 blocks the posterior opening while experiencing the distally directed force and the stabilizing member 112 is positioned against the nose 11 of the patient to produce the proximally directed traction force, the locking member 113 inhibits distal movement of the expandable member 110 away from the posterior naris 14. The stabilizing member 112 can reduce the movement of the catheter assembly 100 that can occur because of the distally directed force on the expandable member 110.

The stabilizing member 112 has a size and shape that inhibits the stabilizing member 112 from entering the nasal cavity 12 of the patient through the anterior naris 13. The stabilizing member 112 has a size greater than the size of the anterior naris 13 so that at least a portion of the stabilizing member 112 cannot be advanced into the nasal cavity 12. That portion that remains external to the nasal cavity 12 can be accessed by the operator so that the stabilizing member 112 can be moved proximally away from the nasal cavity 12 after the treatment is complete. That external portion can also provide the surface of the stabilizing member 112 that pushes against the nose 11 to generate the proximally directed traction force. The stabilizing member 112 can have a diameter and/or width between, for example, 1.6 to 2.6 centimeters (e.g., between 1.75 and 2.5 centimeters, 1.9 centimeters and 2.4 centimeters, or 2.0 centimeters and 2.3 centimeters).

The stabilizing member 112 can be formed of, for example, a disc of a soft material. The stabilizing member 112 can be a monolithic piece of elastomeric or rubber material, such as latex, silicone rubber, or polyurethane. In some cases, the stabilizing member 112 is a hard material coated with the soft material. For example, the stabilizing member 112 can be a rigid polymer, such as polycarbonate or acrylonitrile butadiene styrene, coated with the soft material. An outer soft material on the stabilizing member 112 can improve comfort for the patient when the stabilizing member 112 is pushed against the anterior naris 13.

As shown in FIGS. 2A and 2C, the catheter body 104 further includes a distance marker 116 visible to the operator during the nerve block procedure. The distance marker 116 can be visible such that, as the operator inserts the catheter body 104 into the nasal cavity 12, the operator can approximate a length of the catheter body 104 that has been inserted into the patient. The operator can therefore use the distance marker 116 to approximate relative positions of the opening 108 for the medical fluid delivery, the expandable member 110, and the insertion end 107. In particular, the operator can use the distance marker 116 to determine when the expandable member 110 has been inserted into the nasopharynx 16 of the patient.

The distance marker 116 is positioned at a predetermined distance from the insertion end 107 of the catheter body 104. The distance of the distance marker 116 from the insertion end 107 can be approximately equal to an average distance between the anterior naris 13 and the nasopharynx 16 expected for adult patients. The distance of the distance marker 116 from the insertion end 107 can be approximately between, for example, 8 centimeters and 12 centimeters e.g., between 8.5 centimeters and 11.5 centimeters, 9 centimeters and 11 centimeters, 9.5 centimeters and 10.5 centimeters).

As shown in FIG. 2B, the insertion end region 106 of the catheter assembly 100 includes, in addition to the expandable member 110 and the opening 108, a radiopaque marker 114 and a radiopaque marker 115. The radiopaque marker 114 is positioned on the catheter body 104 distal to the expandable member 110, and the radiopaque marker 115 is positioned on the catheter body 104 proximal to the expandable member 110. The radiopaque markers 114, 115 can be formed of high-density metallic materials that can block radiation, such as x-rays. These metallic materials can include platinum, gold, or other appropriate material that will be distinguished from surrounding human tissue under x-ray fluoroscopy. Viewed under x-ray fluoroscopy, the radiopaque markers 114, 115 demarcate proximal and distal ends of the expandable member 110 such that, during treatment, an operator of the catheter assembly 100 can determine a location of the expandable member 110 relative to surrounding human tissue.

As shown in FIG. 2A, a manipulation end region 118 of the catheter assembly 100 includes an inflation gas port 122 and a medical fluid port 124. Referring to FIG. 2D, which shows a cross-section of the catheter assembly 100 taken along the section line 2D-2D shown in FIG. 2C, the catheter assembly 100 includes a gas lumen 126 and a medical fluid lumen 128. The gas lumen 126 and the medical fluid lumen 128 can be each be defined by, for example, thin-walled tubing extending through the catheter body 104.

The insertion end region 106 further includes openings 117 (shown in FIG. 2B) along the catheter body 104 positioned within the expandable member 110 and distal to the opening 108. Distal to both the openings 117 and the opening 108 is a closed distal wall 129 of the catheter body 104.

During treatment, gas travels through the openings 117 to inflate the expandable member 110. In particular, the gas port 122 can be connected to a gas source. Gas can be delivered from the gas source through the gas lumen 126 to the openings 117 to dispense gas into the expandable member 110. The closed distal wall 129 of the catheter body 104 guides delivery of the gas to the expandable member 110 so that the pressure from the gas can inflate and expand the expandable member 110. The gas port 122 further includes a manually operable gate valve 123 that enables the operator of the catheter assembly 100 to prevent gas from travelling from the gas source to the expandable member 110 and from the expandable member 110 to the gas source.

The delivery of gas from the gas source to the openings 117 and into the expandable member 110 moves the expandable member 110 from the unexpanded state to the expanded state. Removal of gas from the expandable member 110, for example, by opening the gate valve 123, moves the expandable member 110 from the expanded state back to the unexpanded state. In the unexpanded state, the expandable member 110 can be moved proximally from the posterior naris 14 of the nasal cavity 12 and can be removed from the nasal cavity 12.

As described herein, the medical fluid 102 travels through the opening 108 that is positioned proximal to the expandable member 110 to be delivered to the sphenopalatine ganglion 24. The medical fluid port 124 can be connected to a medical fluid source. The medical fluid source includes medication diluted to the desired concentration. When the opening 108 is located within the nasal cavity 12 and the medical fluid 102 is delivered toward the opening 108, the closed distal wall 129 of the catheter body 104 limits delivery of the medical fluid 102 to within the nasal cavity 12. The medical fluid 102 can be delivered through the medical fluid lumen 128 to the opening 108 and then dispensed into the nasal cavity 12. The medical fluid port 124 can include a valve that enables the operator to control the flow of the medical fluid 102 to the opening 108.

Referring back to FIG. 2A, the catheter assembly 100 further includes a hub 130 that serves as an orientation indicator 132. The hub 130 is located on the catheter body 104 in the manipulation end region 118 where the catheter body 104 bifurcates to provide separated sections for the gas port 122 and the medical fluid port 124. The medical fluid lumen 128 and the gas lumen 126 extend through the hub 130 to connect the medical fluid port 124 and the gas port 122, respectively, to the opening 108 and the openings 117, respectively.

The orientation indicator 132 of the hub 130 indicates an angle of orientation of the catheter assembly 100 about the longitudinal axis of the catheter body 104. The orientation indicator 132 can include marks indicating 30-degree increments about the longitudinal axis. As the catheter body 104 rotates, the orientation indicator 132 rotates. Because the orientation indicator 132 rotates with the catheter body 104, it also rotates with the opening 108 defined by the catheter body 104. The opening 108 can be positioned such that a zero-degree demarcation of the orientation indicator 132 corresponds to the direction of the opening 108.

During treatment, the operator of the catheter assembly 100 can observe the orientation indicator 132 to determine an orientation of the opening 108 and to determine the direction that medical fluid will be delivered from the opening 108 into the nasal cavity 12. For the right nasal cavity, the operator can rotate the catheter assembly 100 until the orientation indicator 132 shows a demarcation indicating an orientation between, for example, 30 degrees and 90 degrees (e.g., approximately 60 degrees). Such an orientation of the orientation indicator 132 would indicate that the opening 108 in the right nasal cavity is directed toward the sphenopalatine ganglion 24. For the left nasal cavity, the operator can rotate the catheter assembly 100 until the orientation indicator 132 shows a demarcation indicating an orientation between, for example, 270 degrees and 330 degrees (e.g., approximately 300 degrees). Such an orientation of the orientation indicator 132 would indicate that the opening 108 in the left nasal cavity is directed toward the sphenopalatine ganglion 24.

### Methods of Use

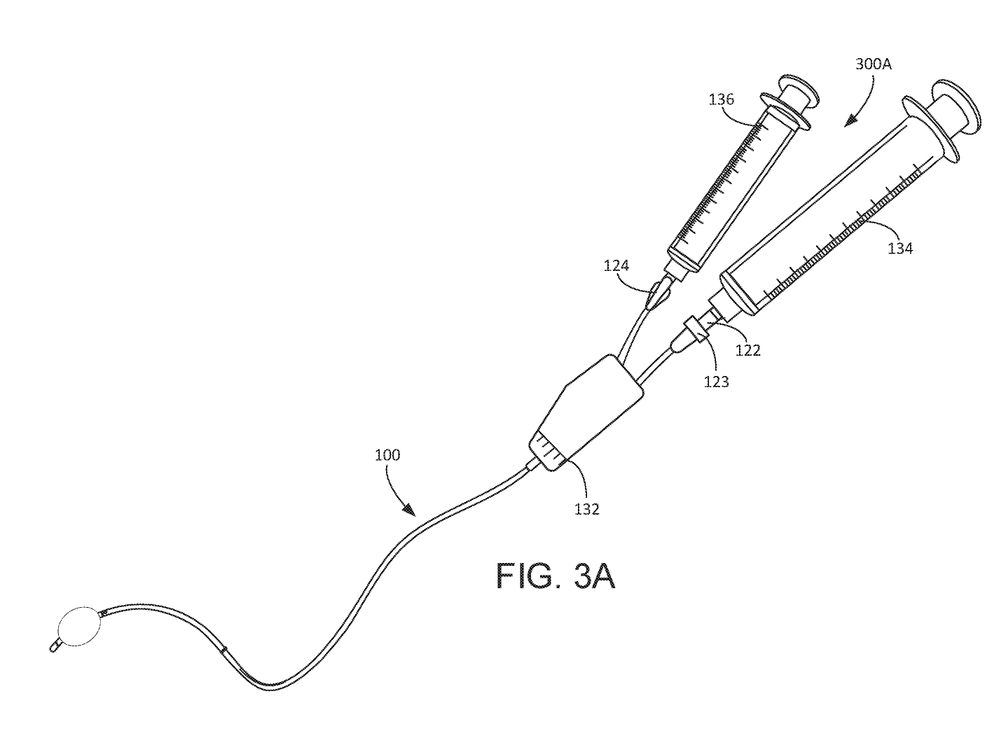

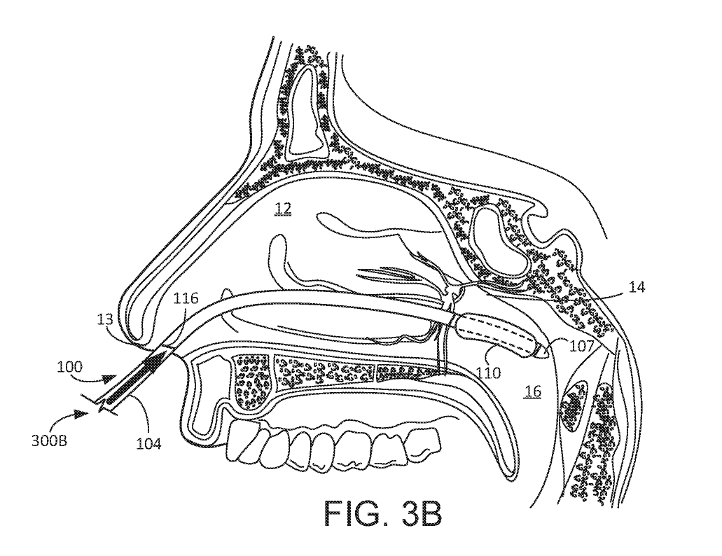

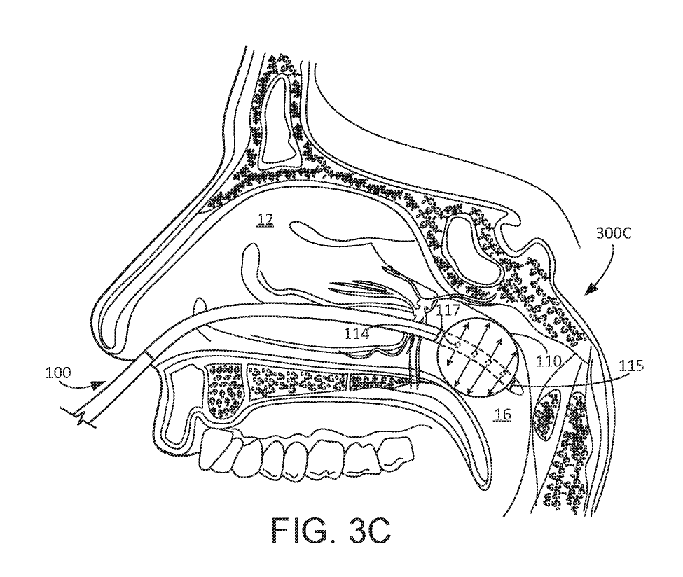

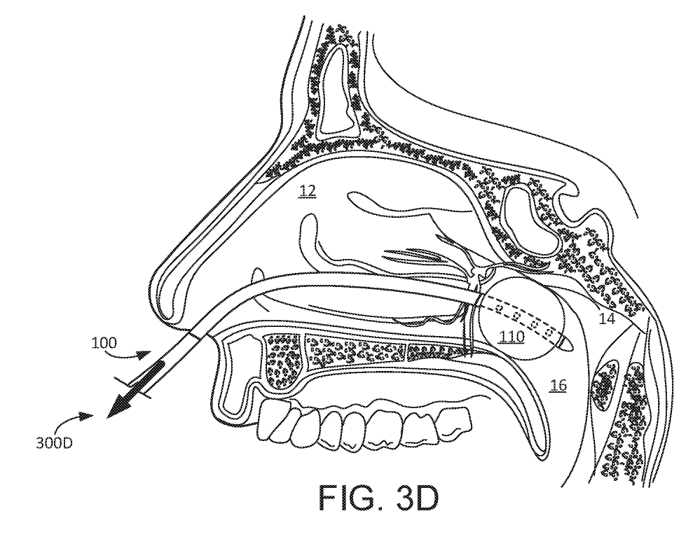

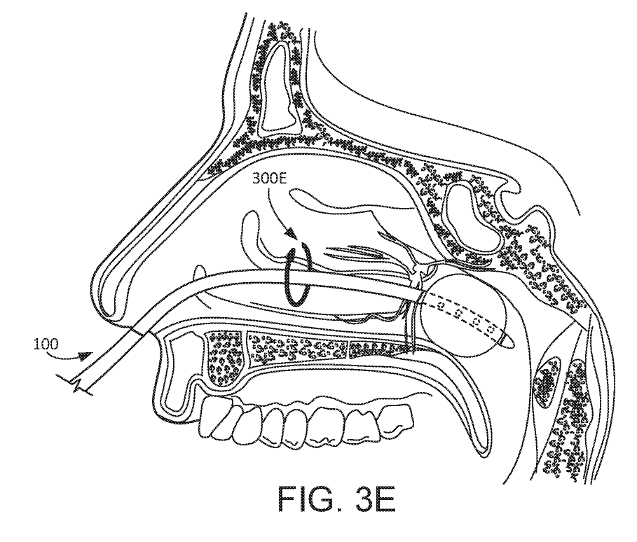

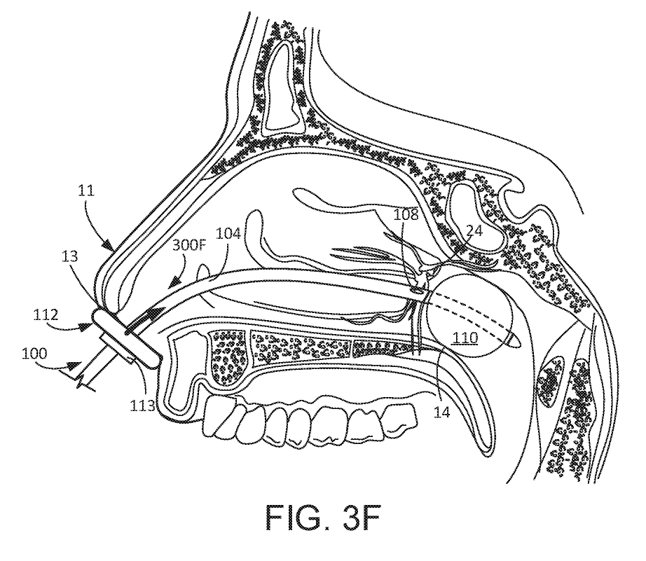

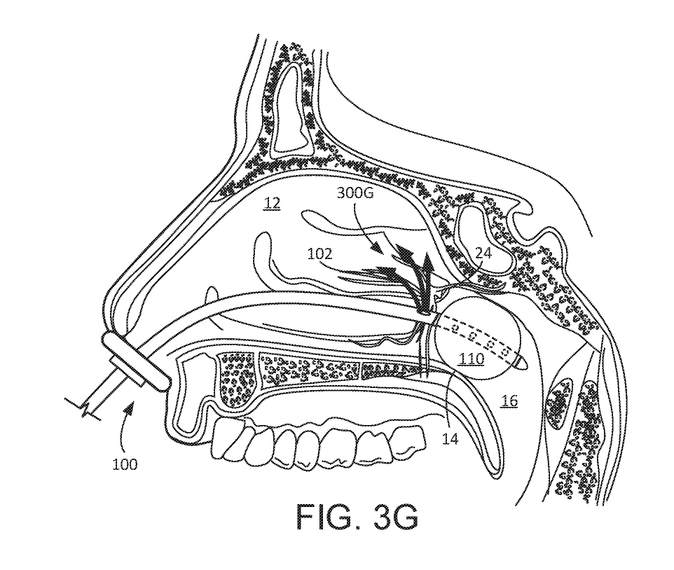

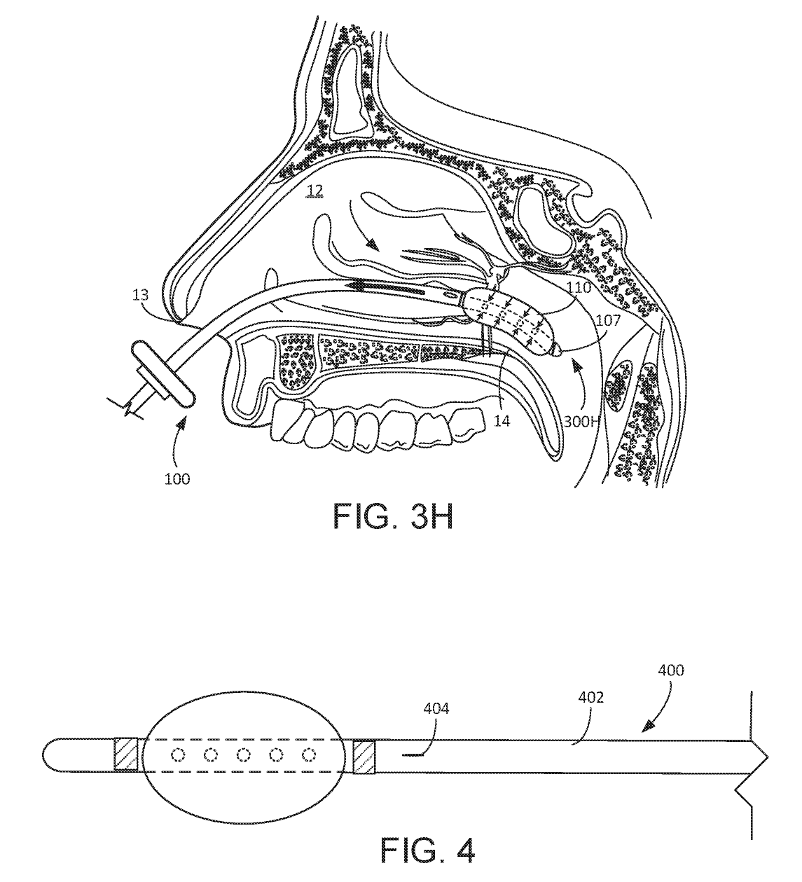

The operator of the catheter assembly 100, as described herein, can use the catheter assembly 100 to perform various medical procedures. The examples shown in FIGS. 3A to 3H depict operations 300A to 300H that can be part of performing a nerve block of the sphenopalatine ganglion. Before performing the operations 300A to 300H to treat a patient, the operator can diagnose patient as having a neurological disease affecting the sphenopalatine ganglion.

At operation 300A depicted in FIG. 3A, the operator connects a gas source 134 to the gas port 122 and a medical fluid source 136 to the medical fluid port 124. When the operator connects the gas source 134 to the gas port 122, the gas source 134 is placed in fluid communication with the gas lumen 126. The gas source 134 can be, for example, a syringe filled with gas. The medical fluid source 136 can be, for example, a syringe filled with a medical fluid.

Before proceeding with inserting the catheter assembly 100 into a patient, the operator can perform an inspection of the nose of the patient. The operator can inspect the nasal cavity 12 for obstructions, inflammation, bleeding, masses, and other abnormalities or contraindications for the nerve block procedure. If the operator determines that the state of the nasal cavity 12 is appropriate for proceeding, the operator can optionally spray the medical fluid containing anesthetic into the nasal cavity 12. The medical fluid can be propelled as a mist that coats portions of the nasal cavity 12 to anesthetize the nasal passages of the patient. The anesthetization of the nasal passage before inserting the catheter assembly 100 can reduce discomfort or pain that the patient could experience during the nerve block procedure. The anesthetic included in the medical fluid can be, for example, lidocaine.

At operation 300B depicted in FIG. 3B, the operator inserts the catheter assembly 100 into a patient. The patient can be in a supine position. The operator inserts the catheter assembly 100 into the nasal cavity 12 and the nasopharynx 16 of the patient such that the expandable member 110 is positioned within the nasopharynx 16. The operator inserts the expandable member 110 through the anterior naris 13, through the nasal cavity 12, and then past the posterior naris 14 into the nasopharynx 16. The operator inserts the expandable member 110 while the expandable member 110 is in the unexpanded state.

As the operator inserts the insertion end 107 of the catheter body 104 into the nasal cavity 12, the operator can observe the distance marker 116 to determine when to stop advancing the insertion end 107 into the patient. When the distance marker 116 reaches the anterior naris 13 of the patient, the operator can stop advancing the insertion end 107. The operator, at this point, has inserted the insertion end 107 of the catheter body 104 approximately 10 centimeters past the anterior naris 13. The expandable member 110, after the operator has advanced the distance marker 116 to a position approximately aligned with the anterior naris 13, is positioned within the nasopharynx 16.

As described herein, the catheter assembly 100 optionally includes the radiopaque markers 114, 115, which also can be used to locate the expandable member 110 within the nasal cavity 12. The operator can observe the position of the radiopaque markers 114, 115 using x-ray fluoroscopy or other appropriate imaging techniques.

After inserting the expandable member 110 into the nasopharynx 16, at operation 300C depicted in FIG. 3C, the operator expands the expandable member 110 in the nasopharynx 16. The operator can open the gate valve 123 to enable gas in the gas source 134 to be delivered through the gas lumen 126 and through the openings 117 into the expandable member 110. The operator can depress the syringe plunger of the gas source 134 so that gas contained within the gas source 134 is delivered to the expandable member 110. The gas from the gas source 134 is delivered through the gas lumen 126 and through the openings 117 and into the expandable member 110 to expand the expandable member 110. In particular, at operation 300C, the expandable member 110 is moved from the unexpanded state to the expanded state in which the expandable member 110 has an expanded volume or size as described herein.

The gas source 134 can deliver, for example, 1 cubic centimeters to 10 cubic centimeters (e.g., 2 cubic centimeters to 9 cubic centimeters, 3 cubic centimeters to 8 cubic centimeters, 4 to 7 cubic centimeters, or approximately 5 cubic centimeters) of gas to the expandable member 110. After the operator has delivered the desired amount of gas to cause the expandable member 110 to expand, the operator can lock the gate valve 123 so that the pressure from the gas is maintained within the expandable member 110. The expandable member 110 therefore remains in the expanded state after the operator releases the syringe plunger of the gas source 134. After the operator expands the expandable member 110 into the expanded state, at operation 300D depicted in FIG. 3D, the operator retracts the expandable member 110. At the end of operation 300C, the expandable member 110 is positioned within the nasopharynx 16, which is distal to the posterior naris 14. At operation 300D, the operator retracts the expandable member 110 so that the expandable member 110, in its expanded state, is wedged into the posterior naris 14. The expandable member 110 is compliant relative to the bony structures of the nasal cavity 12 and the posterior naris 14. As a result, the expandable member 110 can conform to the geometry to the posterior naris 14 and compress when it is pulled against the posterior naris 14.

Furthermore, retracting the expandable member 110 away from the nasopharynx 16 toward the posterior naris 14 applies a force to tissue defining the posterior naris 14. The expandable member 110, while wedged into the posterior naris 14, can block fluid in the nasal cavity 12 from flowing from the nasal cavity 12 into the nasopharynx 16. The operator can continue retracting the expandable member 110 until the operator feels a tactile resistance against the retraction. For example, when the operator feels the tactile resistance, it can indicate to the operator that the expandable member 110 has formed a substantially fluid-tight seal at the posterior naris 14 between the nasal cavity 12 and the nasopharynx 16. The expandable member 110 can, for example, form a fluid-tight seal between 80% and 100% of a periphery of the posterior naris 14. The expandable member 110, after forming the seal with the periphery of the posterior naris 14, can reduce flow of the medical fluid from the nasal cavity 12 into the nasopharynx 16.

At operation 300E depicted in FIG. 3E, the operator rotates the catheter assembly 100 so that the operator can select a direction in which the medical fluid 102 is delivered from the opening 108 (shown in FIG. 3F, depicting the catheter assembly 100 after completion of the operation 300E in FIG. 3E). Also referring to FIG. 2A, the operator can observe the orientation indicator 132 to determine an amount of rotation of the catheter assembly 100. The operator can rotate the catheter assembly 100 such that the orientation indicator 132 (shown in FIG. 3A), as described herein, indicates that the opening 108 is oriented toward the sphenopalatine ganglion 24.

After rotating the catheter assembly 100 to orient the opening 108 toward the sphenopalatine ganglion 24, at operation 300F depicted in FIG. 3F, the operator advances the stabilizing member 112 along the catheter body 104. The operator advances the stabilizing member 112 toward the anterior naris 13 so that the stabilizing member 112 abuts a fleshy portion of the nose 11 of the patient. Referring to FIG. 2C, the operator, after advancing the stabilizing member 112 against the fleshy portion of the nose 11, can activate the locking member 113 to lock the stabilizing member 112 in its position along the catheter body 104.

During this operation, the operator can apply a proximally directed traction force on the catheter assembly 100. For example, the operator can pull the on the catheter assembly 100 so that the expandable member 110 maintains its position within the posterior naris 14. The expandable member 110, by being wedged in the posterior naris 14, can experience a distally directed force opposite of the proximally directed traction force exerted by the operator. The operator's traction force can therefore stabilize the expandable member 110 within the posterior naris 14 so that the expandable member 110 continues blocking the posterior naris 14.

As the operator applies the proximally directed traction force on the catheter assembly 100, the operator can advance the stabilizing member 112 and lock the stabilizing member 112 in its advanced position. By being locked in its position, the stabilizing member 112 is position controlled. The stabilizing member 112, while locked, resists the distally directed force of the expandable member 110 so that the expandable member 110 remains within the posterior naris 14.

Following stabilizing the expandable member 110 within the posterior naris 14, at operation 300G depicted in FIG. 3G, the operator dispenses the medical fluid 102 through the opening 108. The operator dispenses the medical fluid 102 such that the medical fluid 102 contacts the nasal mucosa overlying the sphenopalatine ganglion 24. Because the catheter assembly 100 was rotated to align the opening 108 toward the sphenopalatine ganglion 24 at operation 300E, the medical fluid 102 is delivered toward the sphenopalatine ganglion 24. The operator can dispense, for example, 1 milliliter to 7 milliliters (e.g., 3 milliliters to 5 milliliters, approximately 4 milliliters) of the medical fluid 102. The operator can dispense a varying amount of the medical fluid 102 depending on the concentration of active component within the medical fluid 102.

After dispensing the desired volume of the medical fluid 102, the operator can wait a predetermined amount of time to allow trans-mucosal absorption of the active compound in the medical fluid 102 into the sphenopalatine ganglion 24. In some cases, the operator waits between 5 minutes and an hour (between, e.g., 5 minutes and 45 minutes, 5 minutes and 30 minutes, 5 minutes and 15 minutes, or 5 minutes and 10 minutes, or approximately 7.5 minutes). During the waiting period, because the expandable member 110 is blocking the posterior naris 14, the medical fluid remains within the nasal cavity 12 and tends not to drain into the nasopharynx 16.

The medical fluid 102 can be delivered as, for example, a liquid, a foam, or a combination of liquid and foam. The medical fluid 102 includes the appropriate medication for the procedure, including the active compound (e.g., anesthetic or numbing agent) at the appropriate concentration. The medical fluid 102 can include an alcohol, water, a solvent, or other appropriate compounds for delivery of the active compound.

After waiting the appropriate amount of time for the medication to be absorbed by the sphenopalatine ganglion 24, at operation 300H depicted in FIG. 3H, the operator contracts the expandable member 110 from the expanded state to the unexpanded state. To contract the expandable member 110, the operator can open the gate valve 123 (shown in FIG. 3A) so that the gas contained within the expandable member 110 can be released from the expandable member 110 back into the gas source 134 (shown in FIG. 3A). The operator can then pull the syringe plunger so that the gas in the expandable member 110 is forced back into the gas source 134. After contracting the expandable member 110, the operator can retract the catheter assembly 100 so that the expandable member 110 and the insertion end 107 of the catheter assembly 100 can be removed through the anterior naris 13 of the patient.

Following delivery of the medication and removal of the catheter assembly 100 from the nasal cavity 12, the operator can instruct the patient to clear the nasal cavity 12 of mucus and residual medical fluid. The operator can inspect the nasal cavity for bleeding, swelling, irritation, or other abnormal response to the nerve block procedure.

If the operator does not find a contraindication for proceeding, the operator can repeat operations 300A to 300H for the other nasal cavity. The operations for the other nasal cavity differ in that the opening 108 at operation 300E is oriented appropriately for the particular nasal cavity. In particular, as described herein, the rotational position to direct the opening 108 toward the sphenopalatine ganglion 24 differs depending on whether the opening 108 is located within the right nasal cavity or the left nasal cavity.

### Alternative Implementations

The catheter assembly 100 represents an example of a medical apparatus to be used for a nerve block of the sphenopalatine ganglion. The catheter assembly 100 can be modified to include alternative or additional features. Some features of the catheter assembly 100 may also be omitted. In some cases, these modifications can additionally change the operation of the catheter assembly 100, e.g., the operations 300A to 300H using the catheter assembly 100.

In some examples, instead of being expanded and contracted using gas, the expandable member 110 can be a collapsible mechanical structure that expands and contracts from pull wires attached to one or more points along an internal surface of the expandable member 110. The operator can place a tension force on the pull wires using an external handle or other actuation mechanism. The tension force on the pull wires can cause the mechanical structure to collapse. When the tension force is released, the mechanical structure can return to its original form. The expandable member 110 can initially be in the expanded state, and the operator can actuate the actuation mechanism to place the expandable member 110 in the unexpanded state. To move the expandable member 110 from the unexpanded state to the expanded state, the operator can release the actuation mechanism or drive the actuation mechanism in the reverse direction. In some cases, the expandable member 110 can initially be in the unexpanded state, and the operator can actuate the actuation mechanism to place the expandable member 110 in the expanded state.

In some implementations, the expandable member 110 can include an absorptive component along an outer surface of the expandable member 110. The absorptive component can absorb the medical fluid that may flow along an interface between the expandable member 110 and the posterior naris 14 when the expandable member 110 is blocking the posterior naris 14. The absorptive component can be, for example, a sponge or a fabric.

In some examples, the width of the expandable member 110 is a diameter of a sphere approximated by the volume occupied by the expandable member 110. The expandable member 110 can alternatively occupy a volume approximating an ovoid, a disk, a torus, or other 3-dimensional object that can form a seal along the periphery of the posterior naris 14. In some cases, the expandable member 110, when in the unexpanded state, has a profile that matches the profile of the catheter body 104. The expandable member 110 in the unexpanded state, for example, can have a cylindrical shape, a spherical shape, or other appropriate shape. In some cases, the expandable member 110 can be defined by a length as measured along a longitudinal axis of the catheter body 104 and a width as measured along a transverse axis transverse to the longitudinal axis. The width can be the maximum width along the length of the expandable member. The width can be the maximum width at the midpoint of the length of the expandable member.

While described as a rotating locking mechanism, in some implementations, the locking member is a wedge that that can be pushed against the stabilizing member. The wedge can be wedged between the stabilizing member and the catheter body to increase the friction between the stabilizing member and the catheter body. In some cases, the locking member moves along the catheter body independently from the stabilizing member. The locking member, instead of engaging with the stabilizing member to lock the stabilizing member, engages with itself to lock the locking member along the catheter body. The locking member, when locked to its position along the catheter body, can prevent the stabilizing member from moving proximal past the locking member.

The locking member, in some cases, is located distal to the stabilizing member. A locking member located distal to the stabilizing member can include a locking mechanism that engages with the stabilizing member as described herein. In some implementations, the locking member engages with the anterior naris 13. The locking member can be attached to the stabilizing member and can engage with the anterior naris 13 to generate a force that reduces proximal movement of the stabilizing member. For example, the locking member can be wedged into the anterior naris 13 so that the friction force between the locking member and the anterior naris 13 can counter the force from the expandable member when the expandable member is blocking the posterior naris 14.

In some implementations, the locking member of the stabilizing member is a latch with latching arms that can be expanded within the nasal cavity 12 to engage with side walls of the nasal cavity 12. When expanded, the latching arms move apart to latch onto the side wall of the nasal cavity 12. The stabilizing member has an outer diameter preventing movement of the stabilizing member and the latching arms further distal than necessary for the latching arms to properly engage with the side walls of the nasal cavity 12. The latching arms can also be moved together so that they can be removed from the nasal cavity 12.

In some implementations, instead of including a locking member, the stabilizing member has a tight friction fit with the catheter body. The friction fit between the stabilizing member and the catheter body is sufficiently low such that the operator can manually move the stabilizing member along the catheter body. The friction fit between the stabilizing member and the catheter body is also sufficiently high such that the friction force can resist the distally directed force on the expandable member when the expandable member is retracted into the posterior naris 14. For example, in some cases, the stabilizing member has a geometry approximating a torus that can be slid over the catheter body 104. The friction force can therefore serve as the proximally directed force counter to the distally directed force on the expandable member. An inner diameter of the stabilizing member can equal to or less than a diameter of the catheter body 104. For example, the inner diameter can be 90% to 100% of the diameter of the catheter body 104. The stabilizing member is thus sized to form an interference or friction fit with the catheter body 104 such that movement of the stabilizing member relative to the catheter body 104 occurs when the friction force between the stabilizing member and the catheter body 104 is overcome.

In some implementations, the locking member 113 is a sleeve that can be slid over a collet attached to the stabilizing member 112. When the sleeve engages with the collet, the collet engages with the catheter body 104 to increase the friction between the stabilizing member 112. In some implementations, the catheter assembly 100 does not include the distance marker 116. Instead, the operator can rely on tactile feel of the catheter assembly 100 as the catheter assembly 100 advances into the anatomy of the patient to determine the position of the various components of the catheter assembly 100 within the anatomy. In some cases, the operator instead relies on x-ray fluoroscopy to determine the position of, for example, the radiopaque markers 114, 115 or other radiopaque components of the catheter assembly 100.

While a single distance marker 116 is described, in some cases, the catheter body 104 includes two or more distance markers. Each of the distance markers can be positioned at a different distance and can be labeled with the distance. In some cases, the distance markers are also labeled with patient size or other characteristics that would most likely indicate that, when the particular distance marker is aligned with the anterior naris 13, the insertion end 107 of the catheter body 104 is within the nasopharynx 16. In some implementations, the distance markers are numerical distances appropriately positioned along the catheter body such that they indicate the length of the catheter body that has been inserted into the patient. For example, the distalmost distance marker visible to the operator can indicate the length of the catheter body that has entered the patient.

While described as having radiopaque markers 114, 115, in some implementations, the catheter assembly 100 does not include radiopaque markers. The catheter assembly can include additional distance markers that are visible to the operator when the catheter assembly is inserted into the patient.

In some implementations, the outer surface of the expandable member 110 includes radiopaque markers. As the expandable member 110 expands, the operator can observe the radiopaque markers on the outer surface of the expandable member 110 to determine when to stop expansion of the expandable member 110. The outer surface of the expandable member 110 can include at least two radiopaque markers positioned on opposite sides of the outer surface of the expandable member 110. The outer surface can include several pairs of radiopaque markers whose distances can be measured under x-ray fluoroscopy from different views and orientations of the catheter assembly 100. The measured distance can correspond to a maximum diameter or width of the expandable member 110, and based on the measured distance, the operator can determine a size of the expandable member 110 as it is expanded into its expanded state.

The manipulation end region 118 of the catheter assembly 100 can include additional ports. In some implementations, the catheter assembly 100 include a port for irrigation fluid. The catheter assembly 100 can include an additional opening that enables delivery of the irrigation fluid to the nasal cavity 12 so that the nasal cavity 12 can be moistened. As described herein, the nasal cavity 12 can also be moistened with a separate spray that the operator administers before inserting the catheter assembly 100. That spray can include an anesthetic to anesthetize the nasal passages and improve patient comfort.

While the gas has been described to deliver through the gas lumen 126 and the medical fluid 102 has been described to be delivered through the medical fluid lumen 128, in some cases, the medical fluid 102 is delivered through a lumen defined by the catheter body 104. In some implementations, the gas is delivered through a lumen connected to an opening (e.g., one of the openings 117 within the expandable member 110). In such implementations, the portion of the catheter body 104 within the expandable member 110 may include a single opening connected to the gas lumen 126. While described as multiple openings 117, in some cases, the catheter body includes a single opening for gas delivery to the expandable member.

While the opening 108 has been described and shown as a single opening, in some cases, the medical fluid 102 is delivered out of the medical fluid lumen 128 through multiple openings. The openings can be circular. In some implementations, instead of circular openings, the openings can be slots or slits in the catheter body 104.

As shown in FIG. 4A, which depicts an alternative example of an insertion end region 400 of a catheter assembly, a catheter body 402 of the catheter assembly defines a slit 404 through which the medical fluid is delivered. The slit 404 extends along a lateral surface of the catheter body 402. As the medical fluid is delivered through the slit 404, the geometry and dimensions of the slit 404 can cause the medical fluid to be delivered in the form of a spray. The slit 404 can extend along a longitudinal axis of the catheter body 402. In some cases, the catheter body 402 can define two or more slits on the lateral surface of the catheter body 402, and the medical fluid can be delivered from each of the slits. In some cases, instead of extending along the longitudinal axis of the catheter body 402, the slit 404 can extend around a circumference of the catheter body 402.

In some implementations, the opening 108 can be sized and dimensioned to cause the medical fluid to foam as the medical fluid 102 exits the opening 108. The opening 108 can include sharp geometry or abrupt changes in size relative to the medical fluid lumen 128 so that the medical fluid 102 experiences an abrupt change in fluid pressure as the medical fluid 102 is expelled from the opening 108. The abrupt change in fluid pressure can cause foaming of the medical fluid 102 to occur. The foam can result in greater coverage of the nasal cavity 12 during delivery of the medical fluid 102.

In some implementations, the opening 108 can be sized and dimensioned to enable the medical fluid to be delivered through the opening 108 in a viscous form at an adequate flow rate. The medical fluid, for example, can be a viscous liquid, a gel, or a viscous foam. The opening 108 can have a larger diameter than a diameter of the opening 108 if it were configured for a less viscous medical fluid. While described with respect to FIGS. 1, 2A, and 2B as having a diameter between 1 millimeter and 2 millimeter, the opening 108 can have a greater diameter. The diameter of the opening for the viscous medical fluid can be between, for example, 1.5 millimeters and 2.5 millimeters (e.g., approximately 2 millimeters). The catheter body and/or the medical fluid lumen can accordingly have a larger diameter to enable a greater flow rate and to accommodate the larger opening. For example, the catheter body can have a diameter between, for example, 2 millimeters and 4 millimeters (e.g., approximately 3 millimeters, or between French size 6 and 12). In some cases, the opening 108, the catheter body 104, and the medical fluid lumen 128 are configured as described with respect to FIGS. 1 and 2A to 2D, and the viscous medical fluid is delivered at a slower rate at operation 300G.

While described as including demarcations incremented between zero degrees and 360 degrees, the orientation indicator 132 can be a single mark that indicates the orientation of the opening 108. For example, the hub 130 can be an asymmetric component attached to the catheter body 104 such that the orientation indicator 132 is simply the hub 130. The operator, based on the asymmetry of the hub 130, can estimate the orientation of the catheter assembly 100 about the longitudinal axis of the catheter assembly 100. The orientation indicator 132 can be a line, an arrow, or other geometric indicator.

In some implementations, the hub 130 can include a colored marking indicating the rotational position of the opening 108 on the catheter body 104. The hub 130 can include two separate colored markings, each of the colored markings corresponding to a different nasal cavity. For example, one colored marking can indicate a range of orientations. When at least a portion of the colored marking is facing an upward direction and the opening 108 is located within the right nasal cavity, the opening 108 is approximately directed toward the sphenopalatine ganglion 24. The other colored marking can indicate another range of orientations that correspond to appropriate rotational positions to direct the opening 108 toward the sphenopalatine ganglion 24 in the left nasal cavity. In some cases, in addition to or as an alternative to colored markings, the hub 130 can include symbols representing the appropriate rotational positions for the left and right nasal cavities. The symbols can be, for example, an “L” character for the left nasal cavity and an “R” character for the right nasal cavity.

The operations and methods described herein can also include alternative or additional steps and operations. In some cases, the gas source 134 can be a pressurized gas source. Before the pressurized gas source is connected to the gas port 122 at operation 300A, the operator can first close the gate valve 123 to avoid allowing gas to be delivered through the gas lumen 126. By opening the gate valve 123 after connecting the pressurized gas source, the operator enables the pressure of the pressurized gas source to push gas into the expandable member 110. To release the gas at operation 300H, the operator can disconnect the pressurized gas source from the gas port 122 and then open the gate valve 123, thereby releasing the gas contained within the expandable member 110 through the gas port 122.

The expandable member 110, in some examples, can be part of a device separate from the catheter assembly 100. In particular, a catheter system usable for the treatments and processes presented herein can include a medical fluid delivery catheter assembly for delivery of the medical fluid 102 and a blocking catheter assembly for blocking the posterior naris 14 of the nasal cavity 12. The medical fluid delivery catheter assembly includes the medical fluid port 124, the medical fluid lumen 128, the orientation indicator 132, and the opening 108 to dispense the medical fluid 102 into the nasal cavity 12. The blocking catheter assembly includes the stabilizing member 112, the gas port 122, the gas lumen 126, the openings 117, and the expandable member 110.

These catheter assemblies can be separately inserted into the patient. For example, during the operations 300B to 300F, the operator can first insert the blocking catheter assembly and then expand the expandable member 110 of the blocking catheter to block the posterior naris 14. The operator can then stabilize the expandable member 110 within the posterior naris 14 using the stabilizing member 112, as described herein with respect to operation 300F and FIG. 3F. As the blocking catheter assembly does not deliver the medical fluid, the operator need not rotate the blocking catheter assembly to orient it relative to the sphenopalatine ganglion 24. The operator can then insert the medical fluid delivery catheter assembly into the nasal cavity 12 and appropriately reorient the medical fluid delivery catheter assembly so that the opening 108 is directed toward the sphenopalatine ganglion 24. As described with respect to operation 300G and FIG. 3G, the operator can then deliver the medical fluid through the medical fluid delivery catheter assembly toward the sphenopalatine ganglion 24. Following delivery, the operator can first remove the medical fluid delivery catheter assembly from the nasal cavity 12. The operator can then deflate the expandable member 110 of the blocking catheter assembly and remove the blocking catheter assembly from the nasal cavity 12.

While the expandable member 110 has been described to be moved to the expanded state through delivery of gas to the expandable member 110, instead of gas, in some cases, a liquid is delivered to the expandable member 110 to place the expandable member 110 in the expanded state. The liquid can be, for example, saline.

Operation 300D describes retracting the expandable member 110. In some cases, the expandable member 110 can be inflated within the nasal cavity 12 and then advanced into the posterior naris 14. The expandable member 110 can be further expanded after advancing or retracting the expandable member 110 into the posterior naris 14 so that the expandable member 110 occupies a greater volume surrounding the posterior naris 14.

Operation 300E in which the operator rotates the catheter body 104 to orient the opening 108 can occur before the expandable member 110 is expanded. The operator can first rotate the opening 108 toward the sphenopalatine ganglion 24. The operator can then inflate the expandable member 110 and retract the expandable member 110 to block the posterior naris 14 of the nasal cavity 12.

In some implementations, at operation 300F of FIG. 3F, instead of actuating or activating a mechanism to lock the locking member 113 to prevent movement of the stabilizing member 112 along the catheter body 104, the operator can continue advancing the stabilizing member 112 until the locking member 113 wedges into the anterior naris 13. In this case, the locking member 113 can be located distal to an enlarged diameter portion of the stabilizing member 112 so that, while the operator advances the stabilizing member 112 toward the anterior naris 13, the locking member 113 first engages with the nose 11 before the enlarged diameter portion engages with the nose 11.

In some cases, by advancing the stabilizing member 112 toward the nasal cavity 12, the operator can cause the locking member 113 to latch onto an interior portion of the nasal cavity 12. The latching between locking member 113 and the nasal cavity 12 can generate sufficient force to resist the distally directed force on the expandable member 110.

Instead of a targeted delivery of the medical fluid 102 toward the sphenopalatine ganglion 24 as described at operation 300G, the operator can deliver the medical fluid 102 into the nasal cavity 12 and fill the nasal cavity 12 with, for example, a foamed medical fluid containing the anesthetic. The opening 108, when positioned within the nasal cavity 12, can be positioned away or toward the sphenopalatine ganglion 24. The foamed medical fluid can fill the nasal cavity 12. The expandable member 110 can block the posterior naris 14 such that the foamed medical fluid does not drain into the nasopharynx 16. In some cases, the operator can also block the anterior naris 13 with a plug or a sealing member so that the foamed medical fluid does not drain out of the anterior naris 13.

Elements of different implementations described herein may be combined to form other implementations not specifically set forth above. Elements may be left out of the structures described herein without adversely affecting their operation. Furthermore, various separate elements may be combined into one or more individual elements to perform the functions described herein.

Various embodiments discussed herein may be combined with each other in appropriate combinations in connection with the system described herein. Additionally, in some instances, the order of operations (e.g., the operations 300A to 300H described with respect to FIGS. 3A to 3H) may be modified, where appropriate.

What is claimed is:

1. A medical apparatus comprising:
a catheter body having a medical fluid lumen, the catheter body comprising an insertion end region configured to be inserted into a nasal cavity of a patient, a lateral portion of the insertion end region of the catheter body defining a side opening in fluid communication with the medical fluid lumen of the catheter body; and
an expandable member secured to a portion of the catheter body distal to the side opening, the expandable member being configured to block a posterior opening of the nasal cavity of the patient when the insertion end region of the catheter body is positioned within the nasal cavity of the patient and the expandable member is expanded,
wherein the medical fluid lumen is configured to be placed in fluid communication with a source of the medical fluid such that the medical fluid can be passed through the medical fluid lumen and exit the side opening to contact a sphenopalatine ganglion of the patient when the side opening is positioned within the nasal cavity of the patient.

2. The apparatus of claim 1, further comprising a stabilizing member securable to the catheter body and sized and dimensioned to inhibit the stabilizing member from entering the nasal cavity of the patient through an anterior opening of the nasal cavity of the patient, the stabilizing member being positionable against a nose of the patient.

3. The apparatus of claim 2, wherein the stabilizing member is slidable along the catheter body.

4. The apparatus of claim 2, wherein:
the stabilizing member is lockable in a position along the catheter body such that, when the expandable member blocks the posterior opening and the stabilizing member is positioned against the nose of the patient and locked, the stabilizing member inhibits distal movement of the expandable member from the posterior opening.

5. The apparatus of claim 2, wherein the stabilizing member comprises a disc formed of a soft material, the disc having a diameter greater than a diameter of the anterior opening of the nasal cavity.

6. The apparatus of claim 1, further comprising an orientation indicator disposed near a manipulation end of the catheter body and indicative of an angular position of the side opening relative to a longitudinal axis of the catheter body.

7. The apparatus of claim 6, further comprising a hub positioned at the manipulation end of the catheter body and through which the medical fluid lumen extends, the hub comprising the orientation indicator.

8. The apparatus of claim 1, further comprising a radiopaque marker disposed along the catheter body distal to the expandable member.

9. The apparatus of claim 2, further comprising a radiopaque marker disposed along the catheter body proximal to the expandable member.

10. The apparatus of claim 1, wherein the expandable member comprises an inflatable balloon configured to be placed in fluid communication with a source of gas such that the gas can be passed through the catheter body to inflate the inflatable balloon.

11. The apparatus of claim 10, wherein the insertion end region of the catheter body comprises a distal wall that prevents the gas from exiting the catheter body at a position distal to the inflatable balloon and the medical fluid from exiting the medical fluid lumen at a position distal to the side opening.

12. The apparatus of claim 10, wherein the inflatable balloon is inflatable to a volume of at least 1.75 cubic centimeters such that the inflatable balloon, when inflated, is configured to form a substantially fluid-tight seal between the nasal cavity and a nasopharynx of the patient.

13. The apparatus of claim 10, further comprising a gate valve connected to a proximal end of the catheter body, the gate valve being closeable to prevent the gas within the inflatable balloon from escaping the inflatable balloon.

14. The apparatus of claim 1, further comprising a distance marker disposed on the catheter body at a distance from a distal end of the catheter body approximately equal to a distance between an anterior opening of the patient and a nasopharynx of the patient.

15. The apparatus of claim 14, wherein the distance marker is approximately eight to twelve centimeters proximal from the distal end of the catheter body.

16. A method comprising:
blocking a posterior opening of a nasal cavity of a patient; and
delivering a medical fluid through a catheter to a sphenopalatine ganglion of the patient after blocking the posterior opening of the nasal cavity.

17. The method of claim 16, wherein blocking the posterior opening of the nasal cavity comprises expanding an expandable member to block the posterior opening of the nasal cavity.

18. The method of claim 17, further comprising stabilizing the expandable member within the posterior opening of the nasal cavity.

19. The method of claim 18, wherein stabilizing the expandable member within the posterior opening of the nasal cavity comprises stabilizing the catheter against a nostril of the patient.

20. The method of claim 17, further comprising advancing the expandable member into the posterior opening of the nasal cavity before expanding the expandable member.

21. The method of claim 20, wherein advancing the expandable member into the posterior opening of the nasal cavity comprises advancing the catheter into the posterior opening of the nasal cavity.

22. The method of claim 17, wherein expanding the expandable member to block the posterior opening of the nasal cavity comprises expanding the expandable member in a nasopharynx of the patient.

23. The method of claim 22, further comprising, after expanding the expandable member, retracting the expandable member away from the nasopharynx toward the posterior opening to apply a force to tissue defining the posterior opening.

24. The method of claim 23, wherein retracting the expandable member away from the nasopharynx toward the posterior opening causes the expandable member to reduce flow of fluid from the nasal cavity into the nasopharynx

25. The method of claim 23, wherein retracting the expandable member away from the nasopharynx toward the posterior opening causes the expandable member to form a substantially fluid-tight seal around the posterior opening.

26. The method of claim 25, wherein the fluid-tight seal separates the nasal cavity and the nasopharynx of the patient.

27. The method of claim 17, further comprising, after delivering the medical fluid, contracting the expandable member and removing the expandable member from the patient.

28. The method of claim 27, wherein:
expanding the expandable member comprises injecting at least 1.75 cubic centimeters of air into an inflatable balloon, and
contracting the expandable member comprises deflating the inflatable balloon.

29. The method of claim 27, further comprising, exposing the sphenopalatine ganglion to the medical fluid for at least five minutes for trans-mucosal absorption of the medical fluid while the posterior opening is closed.

30. The method of claim 16, wherein delivering the medical fluid through the catheter to the sphenopalatine ganglion of the patient comprises dispensing the medical fluid in a lateral direction from the catheter.

31. The method of claim 30, further comprising, before dispensing the medical fluid in the lateral direction from the catheter, rotating the catheter such that the lateral direction is directed toward the sphenopalatine ganglion.

32. The method of claim 16, wherein the medical fluid comprises an anesthetic.

33. The method of claim 32, wherein delivering the medical fluid to the sphenopalatine ganglion of the patient comprises delivering the anesthetic to the sphenopalatine ganglion to perform a nerve block of the sphenopalatine ganglion.

34. The method of claim 16, wherein delivering the medical fluid comprises delivering a spray of the medical fluid.

35. The method of claim 16, wherein the medical fluid comprises a foam.

36. The method of claim 16, wherein the medical fluid comprises a gel.
